(19) United States
(12) Patent Application Publication
(10) Pub. No.: US 2018/0049695 A1
(43) Pub. Date: Feb. 22, 2018

(54) Body Fat Index

(71) Applicant: Melvin Hector, JR., Tucson, AZ (US)

(72) Inventor: Melvin Hector, JR., Tucson, AZ (US)

(21) Appl. No.: 15/676,952

(22) Filed: Aug. 14, 2017

Related U.S. Application Data
(60) Provisional application No. 62/376,090, filed on Aug. 17, 2016.

(51) Int. Cl. A61B 5/00

(52) U.S. Cl. CPC A61B 5/4872; A61B 5/7278

(57) ABSTRACT

A method of analyzing a subject's demographic information and a scan of the subject's body and providing a body fat index for the subject is provided. A computer accessible data file is provided with a collection of a species' body type images, demographic information associated with each body type image, and a body fat index associated with each body type image. A processor receives a scanned image of a subject and individual demographic information from that subject; processes the individual demographic information to identify in the data file the body type images that correspond to the demographic information of that subject, and then processes the scanned image of that particular subject against the body type images identified as corresponding to the demographic information to determine the body type image that best matches the scanned image of the subject The processor then produces output including the body fat index for that body type image by processing the scanned image of the subject against the body type images identified from the demographic information. This will provide an affordable, reproducible, readily available method for a potentially large audience of interested prospects to accurately estimate the percentage of body fat of subjects for their particular use.

### RELATED APPLICATION/CLAIM OF PRIORITY

This application is related to and claims priority from U.S. provisional application Ser. No. 62/376,090, filed Aug. 17, 2015, which provisional application is incorporated by reference herein

### INTRODUCTION

The present invention relates to a new and useful paradigm in determining and using a body fat index, that takes into consideration different subject (e.g. human, animal) body types, demographic information for each body type, and the body fat indicators associated with those different subject body types, and produces a body fat index for a scanned image of a particular subject, based on demographic information for that subject, and the imagery provided by their scanned image.

In physical fitness, body composition is used to describe the percentages of fat, bone, water and/or muscle in human bodies. Because muscular tissue is denser than fat tissue, two people of equal height and body weight may look completely different from each other because they have a different body composition.

Body composition (particularly body fat percentage) can be estimated in several ways. The most common method is by using calipers to measure the thickness of subcutaneous fat in multiple places on the body. This includes the abdominal area, the subscapular region, arms, buttocks and thighs. These measurements are then used to estimate total body fat with a margin of error of approximately four percentage 44 points.

Another way of determining body composition uses the fact that the overall density of the body (Db) can be calculated from its mass and volume (Db =mass/volume). The mass of the body is found by simply weighing a person on a scale. The volume of the subject's body is easily and accurately determined by completely immersing a subject in water and calculating the volume of water from the weight of water that is displaced (via “underwater weighing”). It should be noted that with the underwater weighing method, the three dimensional form of the body being weighed would be a significant factor in the volume of water displaced, and in determining the body composition of the subject.

Another method is bioelectrical impedance analysis (BIA), which uses the different resistance of electrical flow through fat tissue in the body to estimate body fat.

Assessment of somatic (skeletal) protein can be determined by simple measurements and calculations including mid arm circumference (MAC), mid arm muscle circumference (MAMC), and creatinine height ratio (CHI). Creatinine height ratio is calculated as 24 hour urine creatinine multiplied by 100 over the expected 24 hour urine creatinine for height. This calculation results in a percentage which can indicate protein status, and by implication percentage of muscular composition.

Body composition measurement with Whole-Body Air Displacement Plethysmography (ADP) technology. A technique for measuring body composition has been developed using the same principles as underwater weighing. The technique uses air, as opposed to water and is known as Whole-Body Air Displacement Plethysmography (ADP). Subjects enter a sealed chamber that measures their body volume through the displacement of air in the chamber. Body volume is combined with body weight (mass) in order to determine body density. The technique then estimates the percentage of body fat and lean body mass (LBM) through known equations (for the density of fat and fat free mass).

Body composition measurement with Dual energy X-ray absorptiometry (DEXA) is used increasingly for a variety of clinical and research applications. DEXA Scan is a medical grade test and considered the Gold Standard in body composition testing, over 99% accurate. Total body or estimated total body scans using DEXA give accurate and precise measurements of BMD and body composition, including bone mineral content (BMC), bone mineral density (BMD), lean tissue mass, fat tissue mass, and fractional contribution of fat.

These measurements are reproducible, making them useful (but expensive) for monitoring potential drug distribution in different tissues for pharmaceutical therapy, nutritional or exercise intervention, sports training and or other body composition altering programs. They are also fast, simple, non-invasive, and expose the subject to a level of x-rays less than that of a cross-country flight.

Body Composition is also estimated using cross-sectional imaging methods like magnetic resonance imaging (MRI) and computed tomography (CT). Since MRI and CT give the most precise body composition measures to-date, many pharmaceutical companies are very interested in this new procedure to estimate body composition measures before and after drug therapy, especially in drugs that might change body composition, or be metabolized differently by different tissues, e.g. fat, muscle, bone, etc.

Ultrasound has also been used to measure subcutaneous fat thickness, and by using multiple points a measurement of body composition can be made. Ultrasound has the advantage of being able to also directly measure muscle thickness and quantify intramuscular fat.

For sports purposes, pharmaceutical reasons, health monitoring reasons, it is clear that the ability to measure body fat composition is really important to a number of entities. The most difficult area for most measurement challenges goes like this: how does one look at a 74 inch male who weighs 260 pounds and estimate their body fat? So, with a BMI, the problem remains: how do you measure the body fat reliably between a body builder that has low body fat and an obese male the same height and weight?

### SUMMARY OF THE PRESENT INVENTION

The present invention provides a new and useful paradigm in determining and using a body fat index, that takes into consideration different subject body types, demographic information for each body type, and the body fat index associated with each of those different body types, and produces a body fat index for a scanned image of a particular subject, based on demographic information for that subject, and the imagery provided by the scanned image. While the applicants method is particularly useful with human subjects, it is also applicable to other subject species (e.g. animals). Thus, in this application reference to a “subject” is intended to encompass humans and other species (e.g. animals) whose body fat index can be determined by the method and steps set forth in this application.

According to applicant's method, a computer accessible data file is provided, having a collection of subject body type images, demographic information associated with each body type image, and a body fat index associated with each body type image. Each body type image is preferably a full body 360 degree 3 D image, and has accuracy comparable to the accuracy that can be provided by an MM scan. A processor receives a scanned image of a subject and individual demographic information provided by (or about in the case of an animal) that subject (height, weight, race, sex, age activity level) and processes the demographic information to identify in the data file (e.g. as a subset) the body type images that correspond to the demographic information of that subject. The processor then determines from the subset the body type image that best matches the scanned image of the subject and produces output including the body fat index for that body type image by processing the scanned image of the subject against the subset of body type images identified from the demographic information.

In the preferred practice of the applicant's method, the collection of body type images are 360 degree 3 dimensional body type images and the scanned image of the subject is a 360 degree 3 dimensional scanned image. In addition, the body fat index associated with each 360 degree 3 dimensional body type image is produced by techniques with accuracy substantially comparable to underwater weighing. The collection of body type images are produced by imaging techniques that have a resolution substantially comparable to MRI imagery.

With the present invention, the fat index for a particular subject that is provided by the present invention can be used in a myriad of ways.
a. For example, a triathlete competitor is concerned about the possibility that he might be overtraining. He is well aware that a body fat percentage too low represents a real disadvantage in a sport that requires such sustained rigorous activity. There is no simple, inexpensive, or easy way to get an accurate body fat determination. He is acutely aware that a BMI with exactly the same data input of height and weight yields inconsistent results, the inability to tell if a 74″ male weighing 260 pounds is morphologically more like an obese subject or a body builder. With the present invention, he can enter basic demographics (height, weight, sex, age, training schedule, race) to acquire the data for an appropriate “BMI”—basic metabolic index—and then uses his easily available phone app to download a 360 degree 3 dimensional pictograph of his body type into the processor of the present invention. The present invention then allows him to instantly estimate, given physiognomy and demographics, a very accurate body fat percentage, which he may then use to adjust his training program toward desired goals. The present invention has just done the work of correlating the demographics of the universe of possibilities of our species, and previously matched it with the 360 degree 3 dimensional body image of any human type that pertains to those demographics, including his own, to accurately provide him his information. He may revisit this program at a later date when his demographics, training habits, or physiognomy change to track his progress.
b. As another example, a pharmaceutical company is preparing to test a new drug for hypertension that is widely distributed in fatty tissue. The fatty tissue composition of an 85 year old can range from 8-40% or higher. Calculating the right dose of an experimental medication for the trial is easily done using a program that has matched her age with her sex, activity level, height, weight, race, and 3 dimensional presentation, so that inadvertent mal-distribution of the trial medication is avoided. Or, with the present invention, when she presents to her pharmacy for review and reconciliation of her usual medications, she can step into a scanner for a few seconds that computes her 360 degree 3 dimensional body appearance, matches it with her demographics on the selected same program and determines that 2 of the 8 medications that she is on should be on a higher or lower dose because of their body fat distribution, a source of common medical error. This issue can be revisited when medications change over time.
c. As still another example, a patient has gradually gained weight insidiously over the last few decades. While he continues to attempt to stay fit, because he is “big-boned”, it is hard for him to believe that it is simply fat. After voicing his concerns to his physician, with the present invention he can step into the physician's office 3 dimensional body scanner, enter his demographics, and instantly receive an estimate of his body fat percentage based on height, weight, sex, age, training schedule, and race that gives him a scientifically accurate representation of his body fat percentage. The present invention also meaningfully points out, from his 360 degree 3D imaging, that his pattern of distribution of body fat, is more an “apple” than a “pear”, warning of the potential eventuality of premature heart disease, and the need for appropriate follow-up.
d. As yet one more example, one particular member, of a certain age, weight, and stage of development, of a species of cattle might be considered to be exemplary. To the extent that diet and environment might be manipulated toward that ideal, body fat content could be predicted and adjusted with information about other cattle that can be directed toward better attainment of that ideal mix of lean and fat measured by a body fat index ascertained in our data bank.

These and other features of the present invention will become apparent from the following detailed description and the accompanying figures.

### BRIEF DESCRIPTION OF THE FIGURES

FIGS. 1A and 1B are schematic illustrations of the components and process steps for providing a body fat index for humans, according to the present invention; and

FIG. 2 is a schematic exemplary showing of the demographic possibilities that can be encompassed by the process of the present invention, and an example of how the process of the present invention would work with an individual human subject with specific demographics.

### DETAILED DESCRIPTION

As described above, the present invention provides a new and useful paradigm in determining and using a body fat index, that takes into consideration different body types, demographic information for each body type, and the body fat indices associated with those different body types, and produces a body fat index for a scanned image (preferably a 360 degree 3 D scanned image) of a particular subject, based on demographic information for that subject, and the imagery provided by the scanned image.

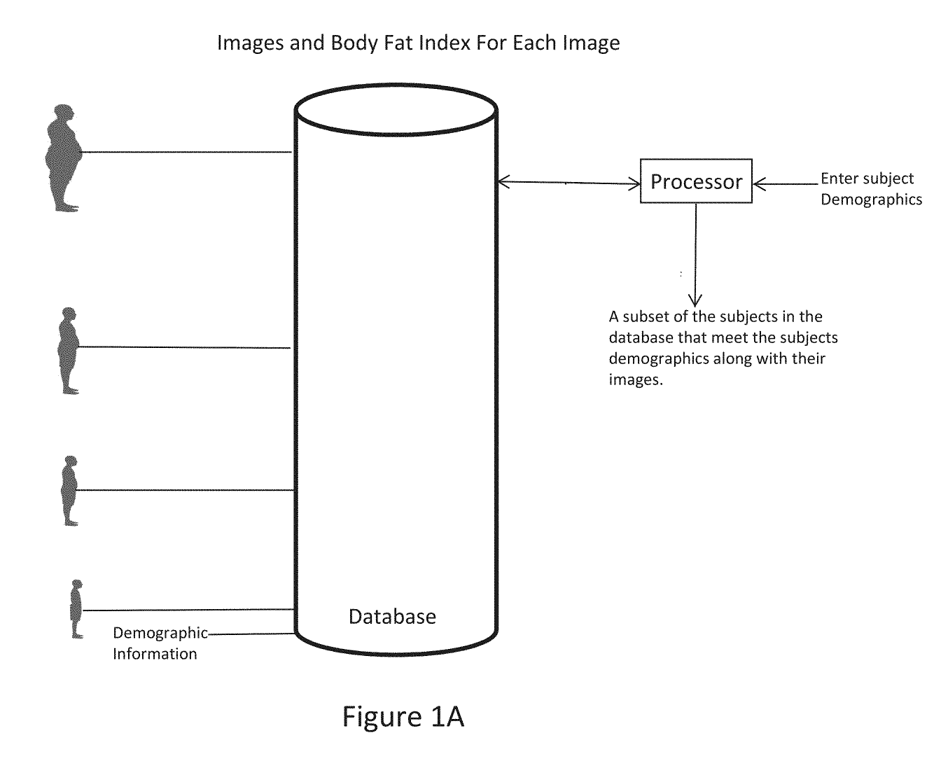

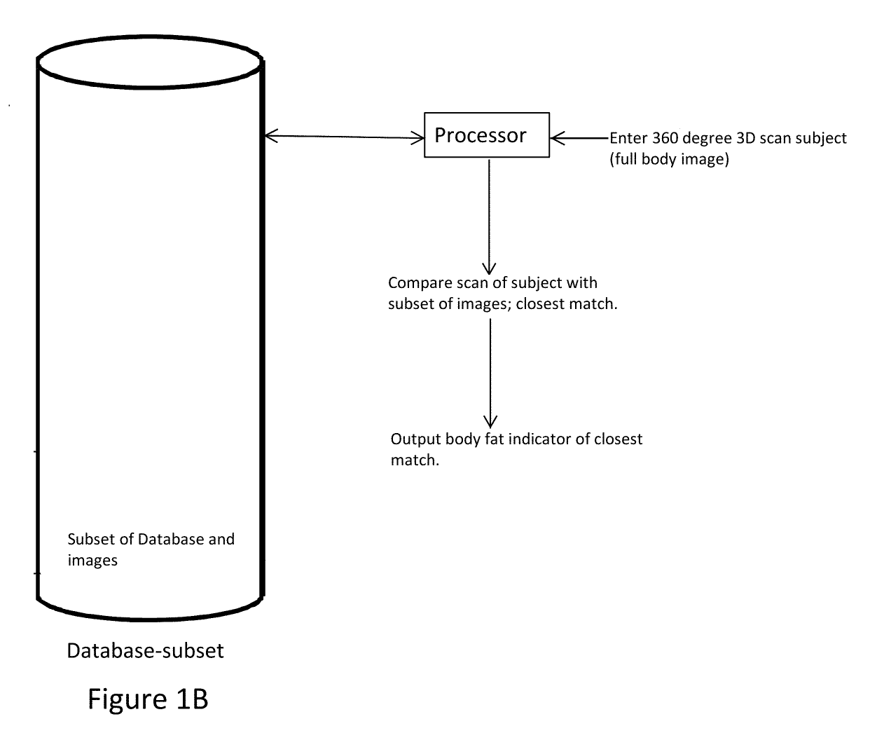

As schematically illustrated in FIGS. 1A and 1B, according to applicant's method, a computer (processor) accessible data file (database) is provided, having a collection of body type images, demographic information associated with each body type image, and a body fat index associated with each body type image. Each body type image is preferably a 360 degree three dimensional full body image, and the image has accuracy comparable to the accuracy that can be provided by an MRI scan. The processor receives a scanned image of a subject (e.g. produced from 360 degree 3D scanning devices such as GIMP (GMU Image Manipulation Program) or imaging from Pelican imaging, and individual demographic information from that subject; and processes the individual demographic information to identify in the data file (e.g. as a subset) the body type images that correspond to the demographic information of that subject. The processor then determines from the subset the body type image that best matches the scanned image of the subject and produces output including the body fat index for that body type image, by processing the scanned image of the subject against the subset of body type images identified from the demographic information.

In the preferred practice of the applicant's method, the collection of body type images are 360 degree 3 dimensional body type images and the scanned image of the subject is a 360 degree 3 dimensional scanned image. In addition, the body fat index associated with each 3 dimensional body type image is produced by techniques with accuracy substantially comparable to underwater weighing. The collection of body type images are produced by imaging techniques that have a resolution substantially comparable to MRI imagery.

With the present invention, the body fat index for a particular subject that is provided by the present invention can be used in a myriad of ways, and, if desired, in the case of a human subject, the privacy of one's own home.

For example, in addition to the uses described above in the Introduction, the present invention can be used by sports teams as a rapid analysis of the body fat information of their athletes, by agencies doing a quick analysis of whether performance enhancing drugs are being used by individuals, by pediatricians counselling growing adolescents regarding safe and appropriate weight loss parameters or concerns, or by ranchers wishing to optimize their herd for their best product.

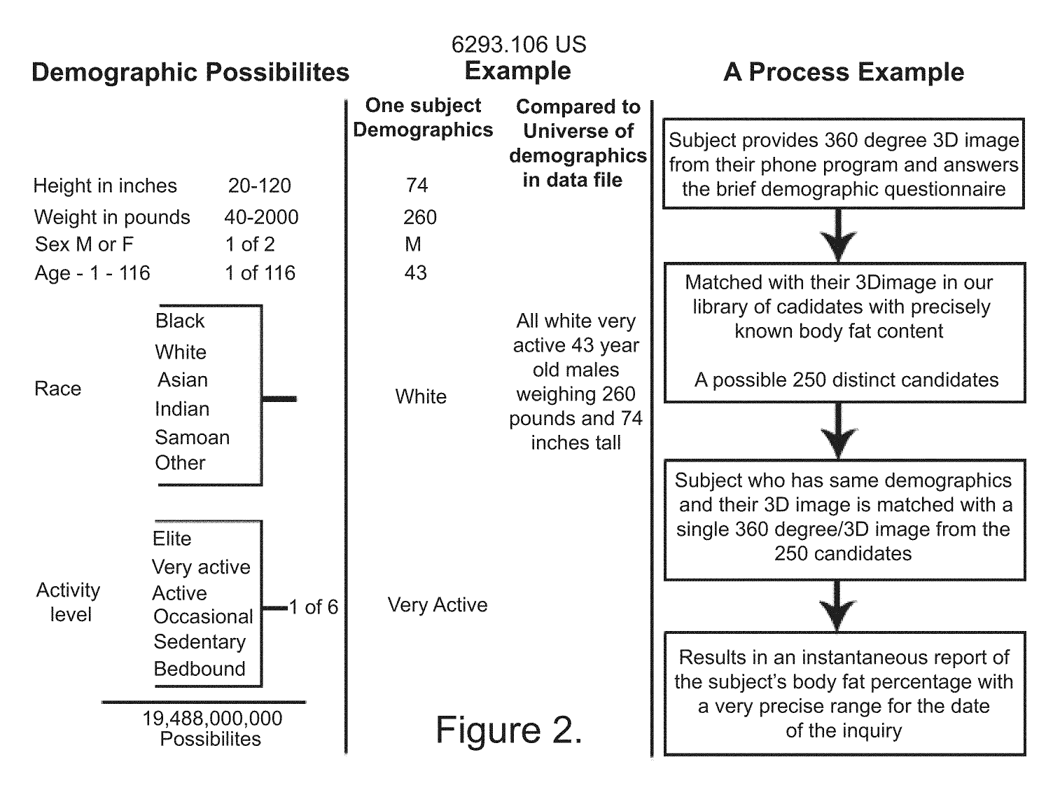

FIG. 2 demonstrates that for the type of demographics that are used in the process of the present invention, the very large amount of possibilities that are associated with those demographics, and then demonstrates that for a single subject (in the example a human subject), with specific demographics, how the process would work to produce a single output that is a very meaningful fat index for that subject. Once the demographics for the subject have identified the subfile (collection) of 360 degree three dimensional images that correspond to the demographics, the comparison of those three dimensional images to the three dimensional image of the subject provides the body fat index for the image that best matches the three dimensional image of the subject.

It is useful to note the importance of using 360 degree 3 dimensional (preferably full body) imagery in the preferred process of the present invention, both in the body images that would populate the data file, and also in the scanned image that would be used in the process once the demographics have been used to provide a collection of body images that fit the demographics. As described herein, two subjects, of substantially similar height and weight, but having different 360 degree three dimensional body forms, may have significantly different body fat measurements, and the process of the present invention uses the different 360 degree three dimensional body forms to determine a meaningful body fat index for each of those subjects.

As will be apparent to those in the art, the development of the images, the body fat index for each image, and the demographic information that would be associated with each image would be collected and expanded on an ongoing basis, so that the data file could enable the practice of the present invention to the fullest extent.

Thus, as seen from the foregoing description, applicant has created a new paradigm for producing a body fat index, and from that description, the manner in which the principles of the present invention can be developed, expanded, and utilized in a myriad of ways will be apparent to those in the art.

1. A method of analyzing a subject's demographic information and a scan of the subject's body and providing a body fat index for the subject, comprising providing a computer accessible data file with a collection of subject body type images, demographic information associated with each body type image, and a body fat index associated with each body type image; providing a processor for receiving a scanned image of a subject and individual demographic information from that subject; processing the individual demographic information to identify in the data file the body type images that correspond to the demographic information of that subject, and then processing the scanned image against the body type images identified as corresponding to the demographic information to determine the body type image that best matches the scanned image of the subject, and producing output including the body fat index for that body type image by processing the scanned image of the subject against the body type images identified from the demographic information.

2. The method of claim 1, wherein the collection of subject body type images are 3 dimensional body type images.

3. The method of claim 2, wherein the collection of subject body images are 360 degree 3 dimensional body image types

4. The method of claim 2, wherein the body fat indicator associated with each 3 dimensional body type image is produced by a technique with accuracy comparable to underwater weighing.

5. The method of claim 1, wherein the scanned image of the subject is a 3 dimensional scanned image of the subject.

6. The method of claim 1, wherein the scanned image of the subject is a 360 degree 3 dimensional scanned image of the subject.

7. The method of any of claims 2 through 6, where the scanned image of the subject has accuracy comparable to an MRI scan.

8. The method of claim 7, wherein the scanned image is a full body image of the subject.

9. The method of claim 8, where the subject is a human subject.

10. The method of claim 7, where the subject is a human subject.

11. The method of claim 6, where the subject is a human subject.

12. The method of claim 5, where the subject is a human subject.

13. The method of claim 4, where the subject is a human subject.

14. The method of claim 3, where the subject is a human subject.

15. The method of claim 2, where the subject is a human subject.

16. The method of claim 1, where the subject is a human subject.
